(19) United States
(12) Patent Application Publication
(10) Pub. No.: US 2026/0009806 A1
(43) Pub. Date: Jan. 8, 2026

(54) METHOD FOR DIRECT QUANTIFICATION OF MULTIPLE APOLIPOPROTEINS IN SERUM

(71) Applicant: SEEGENE MEDICAL FOUNDATION, Seoul (KR)

(72) Inventors: Jong Kee CHUN, Seoul (KR); Je Hyun BAEK, Gwacheon-si, Gyeonggi-do (KR); Won Suk YANG, Bucneon-si, Gyeonggi-do (KR); Hyojin KIM, Incheon (KR); Dong Huey CHEON, Seongnam-si, Gyeonggi-do (KR)

(73) Assignee: SEEGENE MEDICAL FOUNDATION, Seoul (KR)

(21) Appl. No.: 18/993,324

(22) PCT Filed: Jul. 12, 2023
(86) PCT No.: PCT/KR2023/009932

§ 371 Date: Jan. 10, 2025

(30) Foreign Application Priority Data
Jul. 13, 2022 (KR) 10-2022-0086103

(51) Int. Cl. G01N 33/92; G01N 33/68

(52) U.S. Cl. CPC G01N 33/92; G01N 33/6851

(57) ABSTRACT

A method for isolating and/or detecting apolipoproteins from a biological sample by pretreatment with an organic solvent are disclosed. Matrix and internal standard compositions for mass spectrometry of proteins containing ruminant serum are also disclosed. The method, matrix and internal standard compositions not only enable direct quantification of various proteoforms without enzymatic cleavage, but also simple maintenance of the substrate in the sample without removing the target protein by using animal serum as a substrate.

### TECHNICAL FIELD

The present invention relates to a method for the direct quantification of target proteins in human serum, specifically apolipoproteins, by pretreatment with a specific concentration of organic solvent and utilizing non-human animal serum as a substrate and internal standard.

### BACKGROUND ART

Mass spectrometry-based targeted SRM/MRM is one of the most powerful tools for absolute quantification of clinical biomarkers and has been widely used in clinical mass spectrometry for decades. Quantitative proteomics can be used to discover and validate novel protein biomarker candidates, as well as to investigate the mechanisms of disease progression. Traditional antibody-based methods have been routinely used for protein quantification due to their sensitivity, selectivity, and simplicity. But these methods have limitations due to the need for target-specific antibodies and cross-reactivity with other similar proteins. In addition, it is challenging to distinguish sequence differences from their proteoforms that have undergone post-translational modifications (PTMs). Proteoforms are polypeptide variants synthesized from a single gene sequence, but they are also created by a variety of biological processes (e.g., RNA splicing, single nucleotide polymorphisms (SNPs), mutations, or various PTMs), therefore the assays detecting common epitope regions may be limited to quantifying specific proteoforms of functional or clinical importance. Since the bottom-up methods enable high-throughput screening and global proteome analysis, combinations of liquid chromatography and tandem mass spectrometry (peptide-MRM) are widely used for quantitation of multiple targets or massive proteomic analysis. However, these methods suffer from limited sequence coverage and missing for certain peptides containing specific sequence and/or modification due to the high complexity of peptides. In addition, peptide-MRM is time-consuming in that it requires enzymatic protein digestion and complex sample preparation, such as protein denaturation, reduction, or alkylation, despite its ability to rapidly detect analytes on multiple substrates. Therefore, although LC-MS/MS has begun to replace immunoassay methods, there is a growing need for faster and simpler sample preparation methods while maintaining the quantitative accuracy of LC-MS/MS.

Specifically, it is necessary to analyze whole proteins (i.e., proteoforms) without digestion to determine their biological activity or clinical utility. In general, analysis of the intactness of a protein allows for the identification of unique proteoforms and the location of PTMs within each proteoform. Top-down proteomics analyzes intact proteins at the proteoform level, where they are directly ionized and fragmented in a mass spectrometer, allowing for the distinction and characterization of different proteoforms. Quantification of intact proteins at the proteoform level has been steadily developed using quadrupole mass spectrometers, but quantification at the proteoform level is fraught with challenges for clinical applications. Protein precipitation (PPT) efficiently removes most high molecular weight proteins such as serum albumin and immunoglobulins from serum, and organic solvents, salts, and metal ions are widely used as PPT reagents.

On the other hand, apolipoprotein C-III (APOC-III) has been applied as a serum marker for various diseases. It is a low molecular weight glycoprotein (8.8 kDa) associated with apolipoprotein B-containing lipoprotein particles and high density lipoprotein particles, and has been investigated for its potential as a biomarker for disease due to its important role in triglyceride-rich lipoprotein metabolism. Changes in the ratio between glycosylated APOC-III proteoforms or hyper-sialylated APOC-III are responsible for uremia and hypertriglyceridemia. Top-down mass spectrometry and relative quantification methods for APOC-III proteoforms have been investigated, but a valid and efficient absolute quantification method using LC-MS/MS has not been developed.

Therefore, the present inventors sought to develop a protein-MRM method that may quantify each proteoform of APOC-III without digestion of the protein in human serum.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### DISCLOSURE

### Technical Problem

The present inventors have made intensive studies to develop a method for quantifying and identifying full-length proteins with high reliability without digestion, in order to obtain accurate information about the biological activity and clinical utility of the serum proteins. As a result, the present inventors have discovered that pretreatment of biological samples containing the target proteins, more specifically human serum, with a nitrile organic solvent enables selective liquid-phase extraction of the target proteins, more specifically apolipoproteins.

Accordingly, it is an object of the present invention to provide a method for isolating and/or detecting apolipoproteins from a biological sample.

It is another object of the present invention to provide a matrix composition for mass spectrometry of a target protein, comprising ruminant serum.

It is another object of the present invention to provide an internal standard (IS) composition for mass spectrometry of a target protein, comprising ruminant serum.

Other objects and advantages of the present invention will become more apparent from the following detailed description, the appended claims, and the accompanying drawings.

### Technical Solution

In one aspect of this invention, there is provided a method for isolating an apolipoprotein from a biological sample, comprising adding an organic solvent represented by R—CN (wherein R is a straight-chain or branched C₁-C₃ alkyl) to a biological sample comprising an apolipoprotein.

The present inventors have made intensive studies to develop a method for quantifying and identifying full-length proteins with high reliability without digestion, in order to obtain accurate information about the biological activity and clinical utility of the serum proteins. As a result, the present inventors have discovered that pretreatment of biological samples containing the target proteins, more specifically human serum, with a nitrile organic solvent enables selective liquid-phase extraction of the target proteins, more specifically apolipoproteins.

As used herein, the term “isolation of protein” refers to the process of selectively separating the protein of interest contained in a biological sample from other proteins or other impurities other than the protein of interest. Thus, the term “isolation” of a protein is used interchangeably with “extract”, “elute”, “purify”, and “enrich” of a protein.

As used herein, the term “alkyl” refers to a straight-chain or branched saturated hydrocarbon group, and includes, for example, methyl, ethyl, propyl, isopropyl, etc. C₁-C₃ alkyl refers to an alkyl group having an alkyl unit having 1 to 3 carbon atoms, and when the C₁-C₃ alkyl is substituted, the carbon atom number of the substituent is not included. The organic solvent used in the present invention may be, more specifically, propionitrile (where R is C₂ alkyl) or acetonitrile (where R is C₁ alkyl), and more specifically, acetonitrile.

As used herein, the term “biological sample” refers to any material likely to contain apolipoproteins or cells expressing them, cultures thereof, including samples isolated from living organisms (e.g., blood, plasma, serum, saliva, tissues, organs, etc.), materials taken from the environment (e.g., water, air, soil, etc.), or artificially mixed samples. According to a specific embodiment, the biological sample is selected from the group consisting of whole blood, plasma and serum, and most specifically serum.

As used herein, the term “apolipoprotein” refers to a protein that has an ability to transport lipids in blood, cerebrospinal fluid and lymphatic fluid, by binding to lipid such as fat, cholesterol and fat-soluble vitamin, to form lipoproteins. Apolipoproteins are known as an important biomarker and risk factor for lipid-related cardiovascular diseases including atherosclerosis, thus their accurate detection is clinically important. In recent years, apolipoproteins have been measured by immunological methods such as turbidimetric immunoassays (TIA), which have limitations of cross-reactivity, standardization, sensitivity, and multiplexing.

According to a concrete embodiment, the apolipoprotein to be isolated and analyzed in the present invention is selected from the group consisting of ApoA-I, ApoA-II, ApoA-IV, ApoA-V, ApoB, ApoC-I, ApoC-II, ApoC-III, ApoC-IV, ApoD, ApoE, ApoF, ApoL1, ApoL2, ApoL3, ApoL4, ApoL5, ApoL6, Apo(a) and ApoM.

More concretely, the apolipoprotein is selected from the group consisting of ApoL1, ApoM, ApoE, ApoA-II, ApoA-IV, ApoC-II, ApoC-III, ApoD, and ApoF.

Most concretely, the apolipoprotein is ApoC-III.

According to a concrete embodiment, the organic solvent is 40-80 v/v % acetonitrile. More concretely, the organic is 50-70 v/v % acetonitrile, more concretely 55-65 v/v % acetonitrile, and most concretely about 60 v/v % acetonitrile.

In another aspect of this invention, there is provided a method for detecting an apolipoprotein in a biological sample, comprising isolating the apolipoprotein from the biological sample by performing the method for isolating the apolipoprotein of the present invention described above.

The biological samples, organic solvents, and apolipoproteins utilized in the present invention have already been described above in detail and are therefore omitted to avoid undue redundancy.

As used herein, the term “detection” refers to the process of determining the presence of an analyte in a sample. Thus, the term “detection of an apolipoprotein” encompasses all processes for obtaining direct and/or indirect information to determine the presence of the target protein, such as detection or amplification of a nucleic acid molecule encoding an apolipoprotein; immunological analysis using an antibody that specifically recognizes an apolipoprotein or an antigen-binding fragment thereof; and mass spectrometry to detect a mass value corresponding to the full-length protein of an apolipoprotein or a truncated peptide thereof.

The method of the present invention is capable of not only absolute quantification of apolipoproteins in a sample, but also direct identification and quantification of various proteoforms with different glycosylation patterns without enzymatic cleavage. Therefore, the term “detection” is used interchangeably with “quantification” or “identification of proteoforms”.

According to a concrete embodiment, the “detection” is performed by mass spectrometry on the apolipoprotein isolated by the method of the invention described above.

As used herein, the term “mass spectrometry (MS)” refers to the identification of an analyte through its mass value and, more specifically, to an analytical technique that predicts the structure of an analyte based on its mass-to-ion ratio (m/z). MS analysis is typically performed through a step of ionization, which causes the analyte to become charged; and a step of detecting the mass value of the charged material to calculate the m/z value. Specifically, said mass spectrometry may be performed by for example, but not limited to, MALDI-TOF (Matrix-Assisted Laser Desorption/Ionization Time of Flight), SELDI-TOF (Sulfface Enhanced Laser Desorption/Ionization Time of Flight), ESI-TOF (Electrospray ionization time-of-flight), LC-MS (liquid chromatography-mass spectrometry) or LC-MS/MS (liquid chromatography-mass spectrometry/mass spectrometry).

According to a concrete embodiment, the apolipoprotein is ApoC-III, and the method is performed via multiple reaction monitoring (MRM).

MRM, also known as selective reaction monitoring (SRM), is an MS method using triple quadrupole where a first mass filter (Q1) selectively delivers the mother ions from the ion fragments generated by the ionization source to the collision tube, the mother ions arriving at the collision tube are then fragmented by the internal collision gas to generate daughter ions, which are sent to the second mass filter (Q2), and only proteins with specific mother ions and specific daughter ions corresponding to the mass value of the target protein are separated, thereby enabling highly selective and sensitive analysis.

According to concrete embodiment, the method of the present invention does not comprise digestion of the apolipoprotein. The method of the present invention does not include the digestion of full-length proteins by enzymes, which is inevitably included in bottom-up mass spectrometry methods conventionally applied for high-throughput screening and global proteome analysis, thereby enabling complete analysis without limiting sequence coverage and without missing information such as specific variants of target peptides. Therefore, the present invention can be used as a protein-MRM method that can identify and quantify each protein type of apolipoprotein without digestion and cleavage.

In another aspect of this invention, there is provided a matrix composition for mass spectrometry of a target protein in a biological sample comprising serum of a non-human animal as an active ingredient.

In mass spectrometry, the matrix for an analytical sample may actually be mixed in the clinical specimen to be measured. However, in this case, to avoid the distortion of measurement due to the inclusion of the target material to be measured, a matrix free of the target material, or a matrix that has been pretreated to artificially remove the target material should be used. The present inventors have found that the use of the non-human animal serum as a matrix for mass spectrometry in detecting apolipoproteins allows for easy retention of the sample's matrix without removing the target material, while maintaining high detection accuracy.

According to concrete embodiment, the non-human animal is a ruminant.

As used herein, the term “ruminant” refers to a mammal having a rumen, also known as a ruminant stomach, and includes animals of the camelidae, cervidae, deeridae, giraffidae, and bovidae families.

According to a concrete embodiment, the mass spectrometry of the target protein is peptide-MRM (multiple reaction monitoring), and the ruminant is a goat.

According to a concrete embodiment, the mass spectrometry of the target protein is protein-MRM, and the ruminant is a bovine.

The present inventors found that in the peptide-MRM method, goat's serum has a similar matrix composition to human serum, while showing differences in the molecular weights of certain proteins, specifically apolipoproteins, making it a suitable matrix composition, whereas in protein-MRM, bovine serum is a more suitable matrix for liquid extraction of the target protein. Therefore, the linearity of the calibration curve may be maintained by using the serum of each of these animals as the matrix when preparing the calibration curve samples.

In still another aspect of this invention, there is provided an internal standard (IS) composition for mass spectrometry of a target protein in a biological sample comprising a serum protein of a non-human animal as an active ingredient.

As used herein, the term “internal standard” refers to a compound added at a constant concentration to a sample under analysis to calibrate the error of an analytical instrument based on the ratio between the analyte signal and the internal signal. The internal standard is primarily used to correct for the loss of analyte during the sample preparation or injection phase.

According to a concrete embodiment, the non-human animal is a ruminant, and more concretely, a goat or a bovine.

According to a more concrete embodiment, where bovine serum is utilized as the non-human animal serum, the internal standard is one or more selected from the group consisting of the proteins listed in Table 2.

According to a more concrete embodiment, where goat serum is utilized as the non-human animal serum, the internal standard is one or more selected from the group consisting of the proteins listed in Table 3.

According to a concrete embodiment, the mass spectrometry may be performed by matrix-assisted laser desorption/ionization time-of-flight (MALDI-TOF), sulfface enhanced laser desorption/ionization time-of-flight (SELDI-TOF), electrospray ionisation time-of-flight (ESI-TOF), multiple reaction monitoring (MRM), triple quadrupole mass spectrometry (QqQ MS), Composition characterized in that it is made using a mass spectrometry method selected from the group consisting of liquid chromatography-mass spectrometry (LC-MS) and liquid chromatography-mass spectrometry/mass spectrometry (LC-MS/MS).

In still another aspect of this invention, there is provided a method of isolating one or more proteins listed in Table 4 from a biological sample, comprising adding to the biological sample an organic solvent represented by R—CN (R is straight chain or ground C₁-C₃ alkyl).

The organic solvents and biological samples utilized in the present invention have already been described above in detail and are therefore omitted to avoid undue redundancy.

### Advantageous Effects

The features and advantages of the present invention are summarized as follows:
(a) The present invention provides a method for isolating and/or detecting apolipoproteins from a biological sample by pretreatment with an organic solvent.
(b) The present invention also provides matrix and internal standard compositions for mass spectrometry of proteins comprising ruminant serum.
(c) The present invention not only enables direct quantification of various proteoforms without enzymatic cleavage, but also simple maintenance of the substrate in the sample without removing the target protein by using animal serum as a substrate. The present invention also ensures objectivity and accuracy of quantification results at minimal cost by using animal serum protein as internal standards instead of conventional synthetic peptides or proteins that require time and cost.
(d) Accordingly, the present invention may be applied as an efficient analytical method for extracting target proteins from a sample with high purity using a streamlined process and providing information about the total amount of all protein types in a sample as well as individual quantitative information about each protein type with different glycosylation patterns.

### BRIEF DESCRIPTION OF DRAWINGS

FIG. 1 shows the effect of various sample preparation methods (ACN liquid extraction method, MTBE method, TFA method, TCA liquid extraction method, filtration, phosphoric acid method) tried in the present invention measured by SDS-PAGE analysis (top) and Western blot analysis (bottom). The criteria for judging the effectiveness of each method was a combination of whether the complexity of the protein was reduced in SDS-PAGE and the amount of the target protein (e.g., APOC-III protein) was maintained in Western blot. The results showed that the method using acetonitrile (ACN) as an organic best satisfied these criteria.

FIG. 2 represents the SDS-PAGE and Western blot analysis showing the results of exploring the optimal concentration of ACN for sample pretreatment. The optimal concentration was determined based on the reduction of protein complexity and consistent extraction of the target protein. For this purpose, the degree of the target protein extraction was analyzed by densitometry, and the total protein amount was quantified by nano-drop method. The calibration value is the detection level of the target protein corrected to the total protein amount. The results showed that the best effect was achieved at a concentration of 60 v/v % of ACN.

FIG. 3 is a graph representing the quantification results of the liquid-extracted proteins according to the concentration of ACN (40-80%). The results are expressed as the average of three replicate measurements for different proteins belonging to the apolipoproteins. As in FIG. 2, a calibration value is the quantitative value corrected to the total protein amount. Quantitative values were calculated by the area of the chromatogram. 42 proteins including most of the apolipoproteins, were best extracted at 60% ACN.

FIG. 4 shows the results of MALDI-TOF and Q-TOF analysis for identification of proteoforms without enzymatic cleavage. The four proteoforms for APOC-III were identified by mother ions analysis (MS1 only) and tandem analysis (both MS1 and MS2).

FIG. 5 shows the results of liquid-phase extraction of serum with 60% ACN for matrix screening by SDS-PAGE analysis. Human, goat, and bovine sera were treated as matrix, with white boxes representing total proteins without pretreatment and grey boxes representing liquid-phase extracted proteins after pretreatment.

FIG. 6 shows the regression curve of the crude sample in Protein-MRM without (FIG. 6a) and with (FIG. 6b) the matrix, respectively. While it falls on the x-axis in the absence of matrix, in the presence of matrix, not only it points towards zero, but the coefficient of determination of the regression curve is also closer to 1.

FIG. 7 shows the proteins detected after liquid extraction of animal serum with 60% ACN for screening of internal standard material. Representative proteins (albumin and apolipoproteins) from the SDS-PAGE analysis of FIG. 5 are shown.

FIG. 8 is a regression curve showing the effect obtained from the use of internal standards on protein quantitation. The regression curves for Peptide-MRM (FIG. 8a) and Protein-MRM (FIG. 8b) show that the coefficient of determination of the corrected value is closer to 1 when an internal standard is present in the sample. A synthetic peptide was used as an internal standard in Peptide-MRM and a single protein was used in Protein-MRM.

### MODE FOR INVENTION

Hereinafter, the present invention will be described in more detail by way of examples. These examples are only for illustrating the present invention in more detail, and it will be apparent to those skilled in the art that the scope of the present invention according to the subject matter of the present invention is not limited by these examples.

### EXAMPLES

### Example 1. Optimization of Sample Preparation Methods

### Choosing a Preprocessing Method

To explore the optimal way to pretreat samples for liquid extraction of specific proteins from serum, we compared organic solvent (ACN, methyl tert-butyl ether) treatment, acid (trifluoroacetic acid, trichloroacetic acid, phosphoric acid) treatment, and filtration (using 30 kD filters). The specific process for each pretreatment method is described below:
1) ACN liquid extraction method: 300 μl of 100% ACN was added to 100 μl of distilled water, and 50 μl of serum was added to reach a final concentration of 60% ACN. After reacted at 4° C. for 30 min and centrifuged (14,000 g, 30 min, 4° C.), and the supernatant was recovered.
2) MTBE method 1: MTBE (methyl tert-butyl ether):methanol:distilled water were mixed with the ratio of 10:3:2.5, and then reacted at 4° C. for 30 min and centrifuged (21,000 g, 30 min, 4° C.), and the supernatant was recovered.
3) MTBE method 2: MTBE:methanol:distilled water were mixed with the ratio of 5:3:1, and then reacted at 4° C. for 30 min, and centrifuged (21,000 g, 30 min, 4° C.), and the supernatant was recovered. The supernatant was mixed with MTBE:methanol:distilled water=5:1:1, centrifuged (1,000 g, 10 min, 4° C.), and the supernatant was recovered.
4) TFA method: 2 μl of serum was mixed with 50 μl of 0.1% TFA and reacted for 15 minutes at 1,500 rpm, room temperature, and then the salt was removed using a C18 filter.
5) TCA liquid extraction method 1:100 μl of serum was mixed with 100 μl of urea buffer (8M urea, 20 mM DTT in HEPES, pH 8.0) and the protein was denatured for 5 minutes. Then 200 μl of 20% TCA was added and the reaction mixture was incubated at 4° C. for 1 hour, followed by centrifugation (16,000 g, 10 min, 4° C.) and then the supernatant was recovered. The supernatant was filtered through an HLB filter to remove salt.
6) TCA liquid extraction method 2: Proceeded in the same way as the ACN liquid extraction method, but using TCA instead of ACN. Concentrations were adjusted in the range of 1-10% or pretreated sequentially with ACN.
7) Phosphoric acid method: 12% phosphoric acid was added to 2 μl of serum to a final concentration of 2%, reacted at 4° C. for 30 min, centrifuged (21,000 g, 30 min, 4° C.), and the supernatant was collected. The supernatant was pretreated with 60% ACN.
8) Filtration method: 100 μl of serum was mixed with 900 μl of 2% ACN, 0.1% FA solution and centrifuged on a 30 kD filter (14,000 g, 30 min to 1 h, 4° C.). Salts were then removed using an HLB filter.

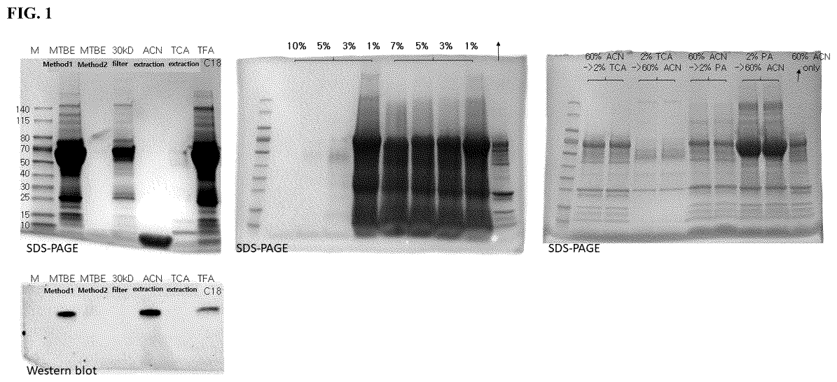

Through the eight methods listed above, the present inventors targeted APOC-III, the most detectable protein in the liquid-phase extraction of serum, and confirmed the pattern of protein bands by SDS-PAGE. In addition, Western blot analysis was performed to determine the degree of protein extraction. As shown in FIG. 1, the present inventors evaluated whether the amount of APOC-III remained constant in the Western blot while reducing the complexity of the protein in the SDS-PAGE, and found that pretreatment with acetonitrile (ACN) among various organic solvents, i.e., method 1) above, was the most efficient for liquid extraction of serum proteins (FIG. 1).

### Optimizing ACN Liquid Extraction Methods

To explore the optimal pretreatment conditions including the concentration of ACN, in the sample preparation method with ACN, the extraction of proteins was evaluated by SDS-PAGE and Western blot analysis at different concentrations (40-80 v/v %), reaction times, and reaction temperatures:
1) ACN concentration: 20 μl of serum was mixed with distilled water and 100% ACN to a final concentration of 40-80% ACN. Sample was reacted for 30 min at 4° C., centrifuged (14,000 g, 30 min, 4° C.) and the supernatant was collected.
2) Reaction temperature: 20 μl of serum was mixed with 40 ul of distilled water and 120 μl of 100% ACN, reacted at room temperature, 4° C., −20° C., and −80° C. for 30 min, centrifuged (14,000 g, 30 min, 4° C.), and the supernatant was collected.
3) Reaction time: 20 μl of serum was mixed with 40 μl of distilled water and 120 μl of 100% ACN, reacted at 4° C. for 30 min and 60 min, centrifuged (14,000 g, 30 min, 4° C.), and the supernatant was collected.
4) Centrifugation conditions: Under the same conditions as above, centrifugation speed (14,000 g, 21,000 g, max) and stopping speed (normal, slow) were compared.

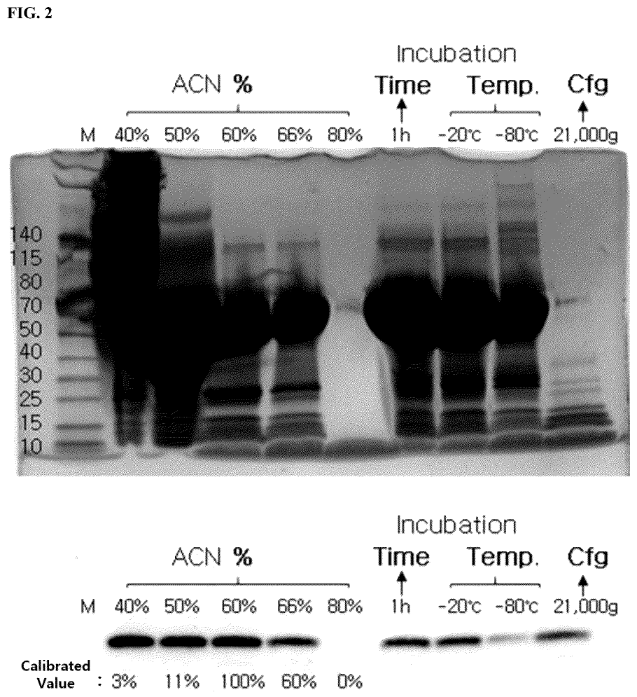

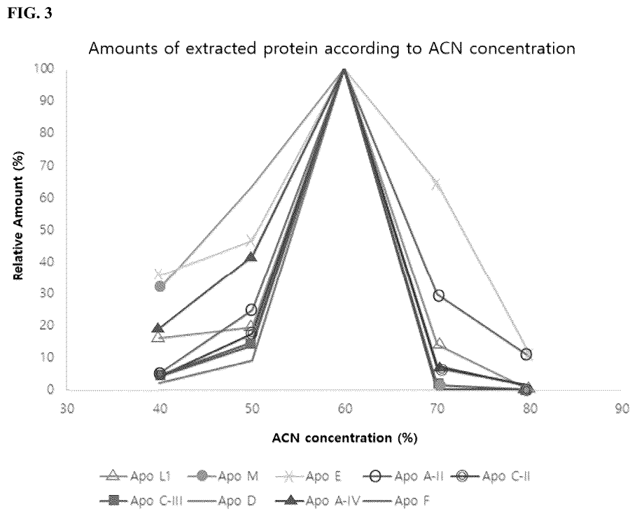

As a result of applying various conditions as described above, it was found that the target protein was extracted most efficiently when the concentration of ACN was 60 v/v %, while the variables of reaction time and reaction temperature were not significant (FIG. 2). Furthermore, the protein fractions obtained using 40-80% ACN were detected by liquid chromatography and dependent mass spectrometry, and the proteins were identified and relatively quantified by non-dependent mass spectrometry. As shown in FIG. 3, various protein families belonging to apolipoproteins showed outstanding extraction efficiency in ACN in the concentration range of 50 to 70%, with the nine measured proteins being best extracted in 60% ACN.

### SDS-PAGE and Western Blot

The pretreated samples were mixed in 5× sample buffer (containing 12.5% 2-mercaptoethanol) and incubated at 95° C. for 10 minutes. Samples were separated by molecular weight on a 4-15% Tris-glycine gel at 200V for 30 minutes. For SDS-PAGE analysis, gels were run in protein fixation buffer containing 50% methanol and 7% acetic acid for 10 min and stained in GelCode Blue solution for 30 min. The residual staining solution was washed with plenty of water.

Western blot analysis was performed by transferring proteins from the gel to a PVDF membrane (20V, 1 hr) and blocking with 5% skim milk or milk powder dissolved in 1×TBS (150 mM NaCl, 20 mM Tris-HCl, pH7.6). Primary antibody (rabbit anti-human APOC-III, 1:1,000) was reacted for 1 hour, washed with 1×TBS-T (1×TBS containing 0.1% tween20 in 1×TBS). The secondary antibody (goat anti-rabbit IgG-HRP, 1:20,000) was reacted for 1 hour, washed with 1×TBS-T. The membrane was immersed in ECL solution and the amount of fluorophore emission was measured with an iBright CL 1000.

### Pretreatment for Protein Identification and Quantification

1) ACN liquid extraction method: 10 μl of serum was mixed with distilled water and 100% ACN (chilled) to a final concentration of 40-80% ACN. The mixture was reacted for 30 min at room temperature, then centrifuged (21,000 g, 30 min, 4° C.) and the supernatant was recovered. The filtered fraction was passed through a C18 filter and dried.
2) Enzymatic cleavage in solvent: 8M urea solution was mixed with the protein sample to a final concentration of 6-8M. 200 mM DTT was mixed to a final concentration of 10 mM and reacted at 37° C., 30 min, 600 rpm. 200 mM IAA was mixed to a final concentration of 15 mM and reacted at 25° C., 1 hour, 0 rpm, in the dark. The urea concentration was diluted with 50 mM Tris-HC1, pH8.0 to a final concentration of 1 M or less. The enzyme used for enzymatic cleavage (Trypsin or Trypsin/Lys-C) was mixed at a 1:50 ratio and reacted at 37° C., 16-24 hours, 900 rpm. 5% formic acid was added to a final concentration of 0.5% and the salt was removed with a C18 column.
3) Orbitrap MS: 200 ng of pretreated sample or an amount of peptide (800 ng or less) calibrated to serum volume was injected into an Ultimate 3000 nanoLC (solvent A was 0.1% formic acid; solvent B was ACN, 0.1% formic acid; columns were a 75 um×2 cm C18 trap column and a 75 um×70 cm C18 analytical column). Peptides were applied to the trap column with solvent A at a rate of 5 μL per minute and analyzed for 90 minutes with a gradient of solvent ratio from 10-40% in solvent B at a rate of 0.35 μL per minute. Orbitrap MS was performed using Q-Exactive HF-X with data dependent analysis (DDA) for protein identification and database building, and data independent analysis (DIA) for relative quantitation with m/z bins. Mass spectrometry data were analyzed with an FDR of 1% or less in the human Uniprot protein database (42,290 protein sequences downloaded on Mar. 22, 2022).

### Example 2. Mass Spectrometry Method for Direct Protein Identification and Absolute Quantification

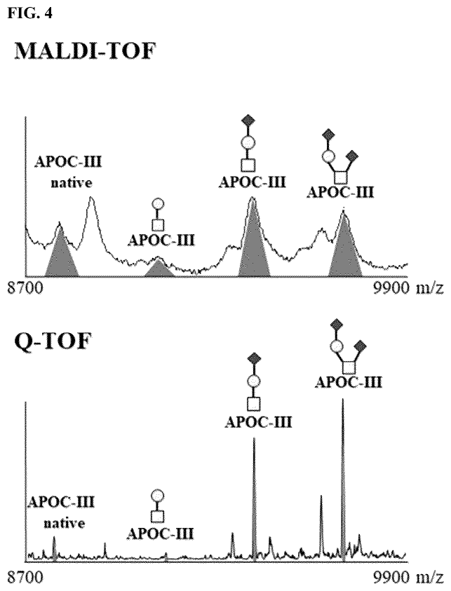

The present inventors sought to perform protein-MRM mass spectrometry that can directly quantify various protein types without enzymatic cleavage. Proteins were extracted from serum with 60% ACN pretreatment according to the method established in Example 1 above, and four isoforms of APOC-III as representative proteins were identified by MALDI-TOF and LC-Q-TOF (FIG. 4).

### MALDI-TOF (Identification)

1 μl of the pretreated sample was spotted onto an MSP 96 metal plate and dried. 1 μl of the matrix solution (20 mg/mL sinapic acid, 0.1% TFA) was spotted on top and dried. The samples were analyzed on a Microflex LT/SH MALDI-TOF with laser 70, gain 7, frequency 66.6 Hz, and 600 shots.

### LC-Q-TOF (Identification)

5 μl of the pretreated sample was injected into a 1260 HPLC (solvent A: 0.1% formic acid; solvent B: ACN, 0.1% formic acid; column: 150×2.0 mm id, 5um Jupiter 300 C4, temperature 40° C.). The analysis was run for 40 minutes at a rate of 0.2 mL per minute with a gradient of solvent ratio from 5 to 60% based on solvent B at a rate of 4% per minute. Q-TOF was performed using a 6545× TQ-TOF with dual JetStreem ESI sources for intact proteins (using MS only) or top-down analysis (using MS1 and MS2). Data were processed by back-squaring MS1 to identify intact proteins or by back-squaring MS2 using the TopPIC program to identify proteins after matching down to fragment ions. Identifications were confirmed by mass values in the spectra and isotopic pattern matching.

### LC-QQQ (Quantitative, Protein-MRM)

5 μl of the pretreated sample was injected into a 1290 UHPLC (solvent A: 0.1% formic acid; solvent B: ACN, 0.1% formic acid; column: 150×2.0 mm id, 5um, Jupiter 300 C4, temperature 40° C.). The analysis was run for 30 minutes at a rate of 0.4 mL per minute with a gradient of solvent ratio from 5 to 60% based on solvent B at a rate of 4% per minute. QQQ was quantified using a 6495 QQQ with a JetStreem ESI source, using the ion source conditions and transitions set below.
1) Optimization of ionization: The conditions were optimized to ensure effective ionization of apolipoproteins, determined by the detected sensitivity (capillary voltage (5500V), ion source gas temperature (110° C.) and velocity (11 L/min), sheath gas temperature (200° C.) and velocity (11 L/min), nebulizer (25 psi), and nozzle voltage (500V) were optimal).
2) Transition Setup: Precursor ions were set up in MRM based on the conditions explored in the high-resolution MS (Q-TOF) and the collision energy (CE) with the fragment ions were set. The resolution was wide (FWHM=1.2).

### LC-QQQ (Quantitative, Peptide-MRM)

20 μl of the pretreated sample was injected into a 1290 UHPLC (solvent A: 0.1% formic acid; solvent B: ACN, 0.1% formic acid; column: 50×3.0 mm id, 2.7 um, Poroshell 20 EC-C18, temperature 40° C.). The analysis was run for 10 minutes at a rate of 0.4 mL/min with a stepwise change in solvent ratio from 5 to 31% based on solvent B. QQQ was quantified using a 6495 QQQ with a JetStreem ESI source, using optimized ionization conditions and transitions. The resolution was unit (FWHM=0.7).

After the ionization optimization and transition setting for protein-MRM analysis as described above, absolute quantitation of four isoforms was performed on the LC-QQQ instrument. The transitions optimized and used in the protein-MRM method and the peptide-MRM method are summarized in Table 1 below, showing information on the best detected parent and fragment ions and fragmentation energies.

TABLE 2

| | | | Theoretical | Q1 m/z | Q3 m/z | Ion | |
|---|---|---|---|---|---|---|---|
| Methods | Digestion | Proteoforms | mass | (charge) | (charge) | type | CE |
| Protein-MRM | No | apoC-III native (ApoC III [symbol] ) | 8759.2180 | 1096.5 (8+) | 549.5 (2+) | y11 | 28 |
| | | | | | 1229.4(7+) | b77 | 26 |
| | | apoC-III-(Gal) [symbol] (GalNAc) [symbol] (APOC-III [symbol] ) | 9124.3502 | 1015.4 (9+) | 1252.9(7+) | y79 | 22 |
| | | | | | 1240.7(6+) | b66 | 20 |
| | | | | | 1278.6(6+) | b68 | 22 |
| | | apoC-III-(Gal) [symbol] (GalNAc) [symbol] (NeuAc) [symbol] (APOC-III [symbol] ) | 9415.4456 | [symbol] 78.6 (8+) | 217.1(1+) | b2 | 36 |
| | | | | | 1252.3(7+) | y79 | 22 |
| | | apoC-III-(Gal) [symbol] (GalNAc) [symbol] (NeuAc)2 (APOC-III [symbol] ) | 9 [symbol] 6.5410 | 1080.2 (9+) | 1252.7(7+) | y79 | 20 |
| | | | | | 1461.4(3+) | b41 | 24 |
| Peptide-MRM | Yes | R.GWVIDGFSSLK.D | 1195.5870 | [symbol] 98.8 (2+) | 854.4(1+) | y8 | 16 |
| | | | | | 9 [symbol] 3.6(1+) | y9 | 16 |
| | | R.GWVTDGFSSLK[13C6, 15N2].D | 1203.6 [symbol] 2 | 602.8 (2+) | [symbol] 62.4(1+) | y8 | 16 |
| | | | | | 961.5(1+) | y9 | 16 |

[symbol]

indicates data missing or illegible when filed

Peptide-MRM and protein-MRM analyses were performed on APOC-III on the same LC-QQQ instrument, and it is confirmed that the three methods including above two and the previously used antibody-based TIA assay were correlated well.

### Example 3. Screening of the Matrix

Human, goat and bovine serum and saline were tested as matrix material for mass spectrometry, and the suitability of each candidate as a matrix were evaluated by SDS-PAGE analysis and mass spectrometry as follows:

### Sample Pretreatment for Matrix Screening

1) SDC (Sodium deoxycholate) digestion (for peptide-MRM analysis): Serum was diluted 20-fold with 1×PBS and then 10 μl was mixed with 10 μl of internal standard (synthetic peptide). 5 μl of 5% SDC was mixed with 25 μl of trypsin/lys-C (1:3.6) and reacted at 37° C., 45 min, 900 rpm. 10 μl of 5% formic acid was added and centrifuged (12,000 g, 10 min, 4° C.) to recover the supernatant. The supernatant was desalted with a C18 column.
2) 60% ACN liquid extraction (for protein-MRM analysis): To 10 μl of serum, 20 μl of distilled water was added and 45 μl of 100% ACN (chilled) was added to make it 60% ACN. After 30 min reaction at room temperature, the sample was centrifuged (21,000 g, 30min, 4° C.), the supernatant was collected and passed through a C18 filter, and the filtered fraction was dried.
3) SDS-PAGE analysis: Pretreated samples were mixed in 5× sample buffer (containing 12.5% 2-mercaptoethanol) and reacted at 95° C. for 10 min. Samples were separated by molecular weight on a 4-15% Tris-glycine gel at 200V for 30 minutes. For SDS-PAGE analysis, the gel was run in a fixing solvent containing 50% methanol, 7% acetic acid for 10 minutes and stained in GelCode Blue solution for 30 minutes. The residual staining solution was washed with plenty of water.

### Quantitative Mass Spectrometry for Matrix Screening (LC-QQQ)

1) Protein-MRM method: 5 μl of the pretreated sample was injected into a 1290 UHPLC (solvent A: 0.1% formic acid; solvent B: ACN, 0.1% formic acid; column: 150×2.0 mm id, 5 um, Jupiter 300 C4, temperature 40° C.). The analysis was run for 30 minutes at a rate of 0.4 mL per minute with a gradient of solvent ratio from 5 to 60% based on solvent B at a rate of 4% per minute. QQQ was quantified using a 6495 QQQ with a JetStreem ESI source, using the ion source conditions and transitions set below. The resolution was wide (FWHM=1.2).
2) Peptide-MRM method: 20 μl of the pretreated sample was injected into a 1290 UHPLC (solvent A: 0.1% formic acid; solvent B: CAN and 0.1% formic acid; column: 50×3.0 mm id, 2.7 um, Poroshell 20 EC-C18, temperature 40° C.). The analysis was run for 10 minutes at a rate of 0.4 mL/min with a stepwise change in solvent ratio from 5 to 31% based on solvent B. QQQ was quantified using a 6495 QQQ with a JetStreem ESI source, using optimized ionization conditions and transitions. The resolution was unit (FWHM=0.7).

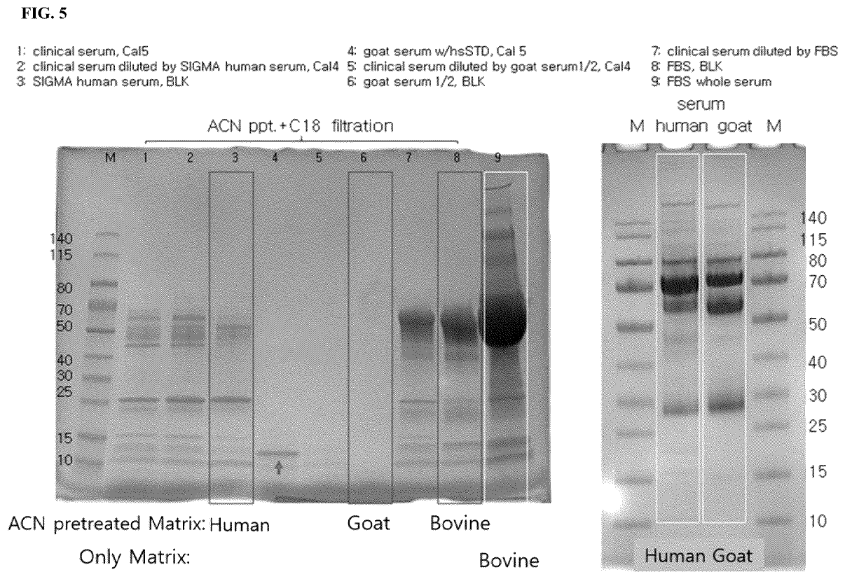

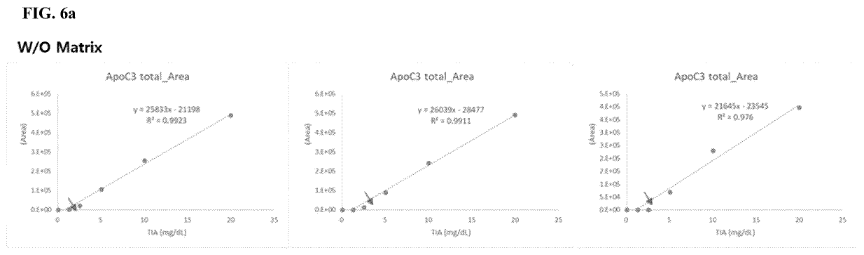

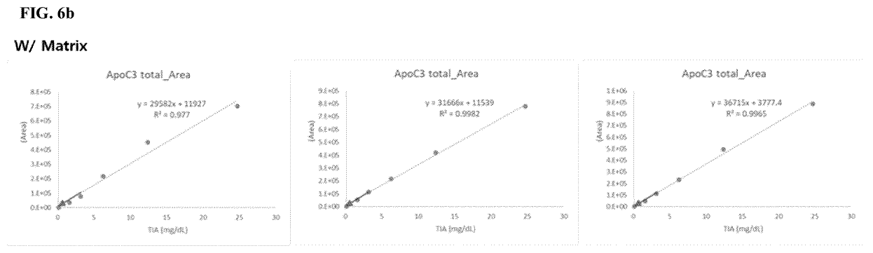

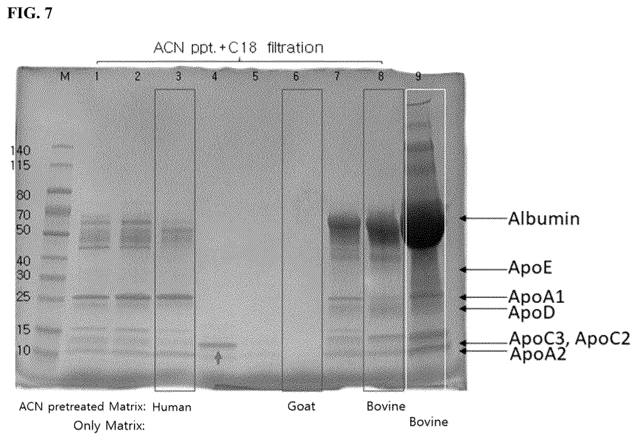

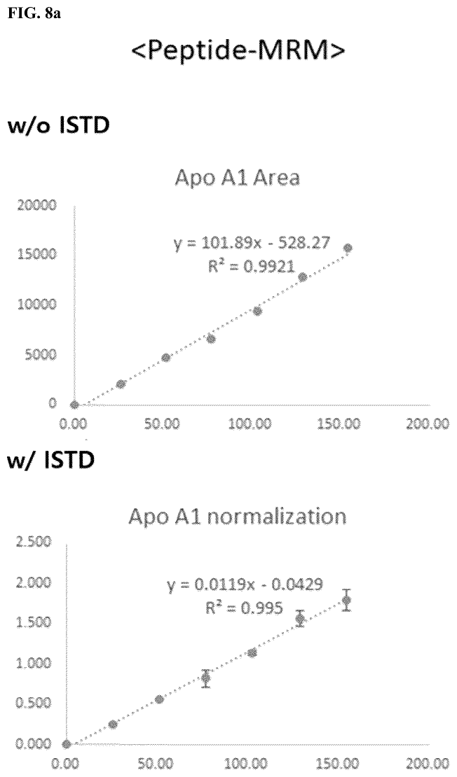

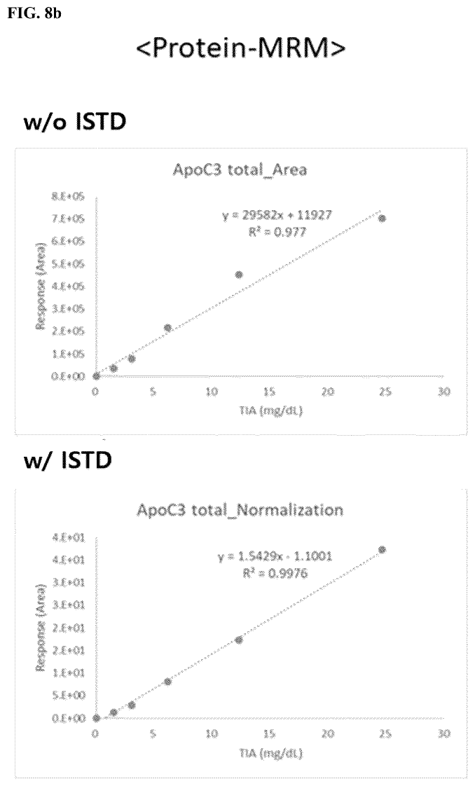

As shown in FIG. 5 (white box), in the peptide-MRM method, goat's serum was identified as a suitable matrix due to differences in specific protein molecular weights while having a similar property to those of human. In contrast, in protein-MRM, goat's serum did not retain the matrix during liquid extraction, and bovine serum was identified as a suitable matrix due to differences in specific protein molecular weights while retaining the matrix (grey box in FIG. 5). Therefore, the use of animal serum as a matrix in the preparation of calibration line samples helped to maintain linearity (FIG. 6).

### Example 4. Search for Internal Standards

Goat and bovine serum were liquid extracted with 60% ACN, and proteins unique to those animals were selected as internal standards by protein identification analysis:

### Sample Preparation for Screening of Internal Standard Candidates

1) 60% ACN liquid extraction method: 10 ul of serum was mixed with 20 μl of distilled water and 45 μl of 100% ACN (chilled) to 60% ACN. After 30 min reaction at room temperature and centrifugation (21,000 g, 30 min, 4° C.), the supernatant was recovered. The filtered fraction was passed through a C18 filter and dried.
2) Enzymatic cleavage in solvent: The protein sample was mixed with 8M urea solution to a final concentration of 6-8M. 200 mM DTT was added to a final concentration of 10 mM and reacted at 37° C., 30 min, 600 rpm. 200 mM IAA was added to a final concentration of 15 mM and reacted at 25° C., 1 hour, 0 rpm, in the dark. The urea was diluted with 50 mM Tris-HCl, pH8.0 to a final concentration of 1 M or less. The cleavage enzyme (Trypsin or Trypsin/Lys-C) was mixed in a 1:20-1:50 ratio and reacted at 37° C., 16-24 hours, 900 rpm. 5% formic acid was added to a final concentration of 0.5% and the salt was removed with a C18 column.

### Identification for Internal Standard and Confirmation of Improved Quantitation

1) Orbitrap MS: 200 ng peptides of the pretreated sample were injected into an Ultimate 3000 nanoLC (solvent A: 0.1% formic acid; solvent B: CAN and 0.1% formic acid; columns: 75 um×2 cm C18 trap column and 75 um×70 cm C18 analytical column). Peptides were applied to the trap column with solvent A at a rate of 5 μL per minute and analyzed for 90 minutes with a gradient of solvent ratio from 10-40% in solvent B at a rate of 0.35 μL per minute. The Orbitrap MS was analyzed by data dependent analysis (DDA) using Q-Exactive HF-X. Mass spectrometry data were analyzed using the Uniprot protein database of goat and bovine (protein sequences downloaded on May 16, 2022, using 77,728 proteins for goat and 47,030 for bovine) with an FDR of 1% or less. Only the proteins with at least two detected peptides were included in the list.
2) LC-QQQ (Protein-MRM method): 5 μl of the pretreated sample was injected into a 1290 UHPLC (solvent A: 0.1% formic acid; solvent B: CAN and 0.1% formic acid; column: 150×2.0 mm id, 5 um, Jupiter 300 C4, temperature 40° C.). The analysis was run for 30minutes at a rate of 0.4 mL per minute with a gradient of solvent ratio from 5 to 60% based on solvent B at a rate of 4% per minute. QQQ was quantified using a 6495 QQQ with a JetStreem ESI source, using the indicated ion source conditions and transitions. The resolution was wide (FWHM=1.2).
3) LC-QQQ (peptide-MRM method): 20 μl of the pretreated sample was injected into a 1290 UHPLC (solvent A: 0.1% formic acid; solvent B: CAN and 0.1% formic acid; column: 50×3.0 mm id, 2.7um, Poroshell 20 EC-C18, temperature 40° C.). The analysis was run for 10 minutes at a rate of 0.4 mL/min with a stepwise change in solvent ratio from 5 to 31% based on Solvent B. QQQ was quantified using a 6495 QQQ with a JetStreem ESI source, using optimized ionization conditions and transitions. The resolution was unit (FWHM=0.7).

TABLE 2
List of proteins detected in bovine serum (52 in total)

| Accession | Description |
|---|---|
| P12763 | Alpha-2-HS-glycoprotein OS = Bos taurus OX = 9913 GN = AHSG PE = 1 |
| | SV = 2 |
| P34955 | Alpha-1-antiproteinase OS = Bos taurus OX = 9913 GN = SERPINA1 PE = 1 |
| | SV = 1 |
| Q5GN72 | Alpha-1-acid glycoprotein OS = Bos taurus OX = 9913 GN = agp PE = 2 SV = 2 |
| P01966 | Hemoglobin subunit alpha OS = Bos taurus OX = 9913 GN = HBA PE = 1 SV = 2 |
| I7CT57 | Gc-globulin OS = Bos taurus OX = 9913 PE = 2 SV = 1 |
| Q3MHN5 | Vitamin D-binding protein OS = Bos taurus OX = 9913 GN = GC PE = 2 SV = 1 |
| P81644 | Apolipoprotein A-II OS = Bos taurus OX = 9913 GN = APOA2 PE = 1 SV = 2 |
| O46375 | Transthyretin OS = Bos taurus OX = 9913 GN = TTR PE = 1 SV = 1 |
| P02769 | Albumin OS = Bos taurus OX = 9913 GN = ALB PE = 1 SV = 4 |
| A0A3Q1MJT2 | Alpha-1B-glycoprotein OS = Bos taurus OX = 9913 GN = A1BG PE = 1 SV = 1 |
| B0JYQ0 | ALB protein OS = Bos taurus OX = 9913 GN = ALB PE = 2 SV = 1 |
| P02081 | Hemoglobin fetal subunit beta OS = Bos taurus OX = 9913 PE = 1 SV = 1 |
| G1K122 | Retinol-binding protein OS = Bos taurus OX = 9913 GN = RBP4 PE = 3 SV = 1 |
| A0A3Q1LVV7 | Fibrinogen alpha chain OS = Bos taurus OX = 9913 GN = FGA PE = 4 SV = 1 |
| A0A452DI66 | Prothrombin OS = Bos taurus OX = 9913 GN = F2 PE = 3 SV = 1 |
| P02453 | Collagen alpha-1(I) chain OS = Bos taurus OX = 9913 GN = COL1A1 PE = 1 |
| | SV = 3 |
| F1MMK9 | Protein AMBP OS = Bos taurus OX = 9913 GN = KIF12 PE = 3 SV = 3 |
| A0A3Q1MGB8 | Protein AMBP OS = Bos taurus OX = 9913 GN = KIF12 PE = 3 SV = 1 |
| V6F9A3 | Apolipoprotein C-III OS = Bos taurus OX = 9913 GN = ApoC3 PE = 3 SV = 1 |
| A0A3Q1M2B2 | Complement C3 OS = Bos taurus OX = 9913 GN = C3 PE = 1 SV = 1 |
| Q58D62 | Fetuin-B OS = Bos taurus OX = 9913 GN = FETUB PE = 1 SV = 1 |
| F1MS32 | Apolipoprotein D OS = Bos taurus OX = 9913 GN = APOD PE = 3 SV = 3 |
| P01045 | Kininogen-2 OS = Bos taurus OX = 9913 GN = KNG2 PE = 1 SV = 1 |
| G3N0V2 | Cytokeratin-1 OS = Bos taurus OX = 9913 GN = KRT1 PE = 1 SV = 2 |
| P01044 | Kininogen-1 OS = Bos taurus OX = 9913 GN = KNG1 PE = 1 SV = 1 |
| A0A0A0MP92 | Serpin A3-7 OS = Bos taurus OX = 9913 GN = SERPINA3-7 PE = 1 SV = 1 |
| A6QNZ7 | Keratin 10 (Epidermolytic hyperkeratosis; keratosis palmaris et plantaris) |
| | OS = Bos taurus OX = 9913 GN = KRT10 PE = 2 SV = 1 |
| Q68RU0 | Ovarian and testicular apolipoprotein N OS = Bos taurus OX = 9913 |
| | GN = ApoN PE = 2 SV = 1 |
| P28800 | Alpha-2-antiplasmin OS = Bos taurus OX = 9913 GN = SERPINF2 PE = 1 SV = 2 |
| A0A3Q1LRP5 | C3/C5 convertase OS = Bos taurus OX = 9913 GN = CFB PE = 1 SV = 1 |
| Q5DPW9 | Cystatin E/M OS = Bos taurus OX = 9913 GN = CST6 PE = 4 SV = 1 |
| A5D7S8 | Fibulin-1 OS = Bos taurus OX = 9913 GN = FBLN1 PE = 2 SV = 1 |
| Q3T0E0 | Copper transport protein ATOX1 OS = Bos taurus OX-9913 GN = ATOX1 |
| | PE = 3 SV = 1 |
| I3PGL3 | Insulin-like growth factor II (Fragment) OS = Bos taurus OX = 9913 |
| | GN = IGF2 PE = 2 SV = 1 |
| Q2KIS7 | Tetranectin OS = Bos taurus OX = 9913 GN = CLEC3B PE = 2 SV = 1 |
| F1MYX2 | Apolipoprotein M OS = Bos taurus OX = 9913 GN = APOM PE = 3 SV = 1 |
| E1B726 | Plasminogen OS = Bos taurus OX = 9913 GN = PLG PE = 3 SV = 2 |
| D4QBB4 | Globin A1 OS = Bos taurus OX = 9913 GN = HBB PE = 3 SV = 1 |
| A0A0A0MPA0 | SERPIN domain-containing protein OS = Bos taurus OX = 9913 |
| | GN = LOC784932 PE = 1 SV = 1 |
| F1N5T0 | Protein CutA OS = Bos taurus OX = 9913 GN = CUTA PE = 3 SV = 1 |
| P15497 | Apolipoprotein A-I OS = Bos taurus OX = 9913 GN = APOA1 PE = 1 SV = 3 |
| A4IFP2 | KRT4 protein OS = Bos taurus OX = 9913 GN = KRT4 PE = 2 SV = 1 |
| F1MLH6 | Calmodulin OS = Bos taurus OX = 9913 GN = CALM PE = 4 SV = 3 |
| P13213 | SPARC OS = Bos taurus OX = 9913 GN = SPARC PE = 1 SV = 2 |
| A0A3Q1LMK6 | Cell growth regulator with EF-hand domain 1 OS = Bos taurus OX = 9913 |
| | GN = CGREF1 PE = 4 SV = 1 |
| G3X6N3 | Beta-1 metal-binding globulin OS = Bos taurus OX = 9913 GN = TF PE = 1 |
| | SV = 2 |
| Q3B7N0 | Cadherin-13 OS = Bos taurus OX = 9913 GN = CDH13 PE = 2 SV = 1 |
| F1N362 | Keratin 84 OS = Bos taurus OX = 9913 GN = KRT84 PE = 3 SV = 2 |
| P13384 | Insulin-like growth factor-binding protein 2 OS = Bos taurus OX = 9913 |
| | GN = IGFBP2 PE = 1 SV = 2 |
| A0A3QIN2U0 | Latent transforming growth factor beta binding protein 1 OS = Bos taurus |
| | OX = 9913 GN = LTBP1 PE = 4 SV = 1 |
| F1NOI3 | Coagulation factor V OS = Bos taurus OX = 9913 GN = F5 PE = 3 SV = 3 |
| A0A3Q1M083 | Protein CutA OS = Bos taurus OX = 9913 GN = CUTA PE = 3 SV = 1 |

TABLE 3
List of proteins detected in goat serum (9 in total)

| Accession | Description |
|---|---|
| P85295 | Albumin (Fragments) OS = Capra hircus OX = 9925 GN = ALB PE = 1 SV = 2 |
| A0A8C2NVN1 | Complement C3 OS = Capra hircus OX = 9925 PE = 4 SV = 1 |
| A0A452E2C8 | Transthyretin OS = Capra hircus OX = 9925 GN = TTR PE = 3 SV = 1 |
| V5KXW5 | Retinol-binding protein OS = Capra hircus OX = 9925 PE = 2 SV = 1 |
| A0A452FX21 | Prothrombin OS = Capra hircus OX = 9925 GN = F2 PE = 3 SV = 1 |
| B3VHM9 | Albumin (Fragment) OS = Capra hircus OX = 9925 PE = 1 SV = 1 |
| A0A452DY37 | Beta-2-microglobulin OS = Capra hircus OX = 9925 GN = B2M PE = 3 SV = 1 |
| A0A452FVB9 | BPI1 domain-containing protein OS = Capra hircus OX = 9925 PE = 4 SV = 1 |
| A0A452GA47 | Cytokeratin-1 OS = Capra hircus OX = 9925 GN = KRT1 PE = 3 SV = 1 |

The straightness of the calibration line was improved when synthetic peptides or single proteins listed above, such as myoglobin and cytochrome C, were used as internal standards.

TABLE 4
Apolipoproteins and other serum proteins maximally extracted from ACN 60%.

| Protein | Genes |
|---|---|
| Immunoglobulin superfamily containing leucine-rich repeat protein | ISLR |
| | UNQ189/PRO215 |
| Coagulation factor XIII A chain (Coagulation factor XIIIa) (EC | F13A1 F13A |
| 2.3.2.13) | |
| Prothrombin (EC 3.4.21.5) (Coagulation factor II) | F2 |
| Angiotensinogen (Serpin A8) | AGT SERPINA8 |
| Complement C3 (C3 and PZP-like alpha-2-macroglobulin domain- | C3 CPAMD1 |
| containing protein 1) | |
| Cystatin-C (Cystatin-3) (Gamma-trace) | CST3 |
| Insulin-like growth factor II (IGF-II) (Somatomedin-A) | IGF2 PP1446 |
| Fibrinogen alpha chain | FGA |
| Leucine-rich alpha-2-glycoprotein (LRG) | LRG1 LRG |
| Retinol-binding protein 4 (Plasma retinol-binding protein) (PRBP) | RBP4 PRO2222 |
| (RBP) | |
| Alpha-1-acid glycoprotein 1 (AGP 1) (Orosomucoid-1) (OMD 1) | ORM1 AGP1 |
| Alpha-2-HS-glycoprotein (Alpha-2-Z-globulin) (Ba-alpha-2- | AHSG FETUA |
| glycoprotein) | PRO2743 |
| Phosphatidylcholine-sterol acyltransferase (EC 2.3.1.43) | LCAT |
| Serine protease 1 (EC 3.4.21.4) (Anionic trypsin I) | PRSS1 TRP1 TRY1 |
| | TRYP1 |
| Serum amyloid A-1 protein (SAA) | SAA1 |
| Coagulation factor V (Activated protein C cofactor) | F5 |
| Insulin-like growth factor-binding protein 2 (IBP-2) | IGFBP2 BP2 IBP2 |
| Insulin-like growth factor-binding protein 4 (IBP-4) | IGFBP4 IBP4 |
| Serum paraoxonase/arylesterase 1 (PON 1) (EC 3.1.1.2) (EC 3.1.1.81) | PON1 PON |
| (EC 3.1.8.1) | |
| Peroxiredoxin-2 (EC 1.11.1.24) (Natural killer cell-enhancing factor B) | PRDX2 NKEFB |
| (NKEF-B) (PRP) | TDPX1 |
| Trypsin-3 (EC 3.4.21.4) (Brain trypsinogen) (Mesotrypsin) | PRSS3 PRSS4 TRY3 |
| (Mesotrypsinogen) (Serine protease 3) | TRY4 |
| Beta-2-microglobulin | B2M CDABP0092 |
| | HDCMA22P |
| Thymosin beta-4 (T beta-4) (Fx) | TMSB4X TB4X |
| | THYB4 TMSB4 |
| Galectin-3-binding protein (Basement membrane autoantigen p105) | LGALS3BP M2BP |
| (Lectin galactoside-binding soluble 3-binding protein) | |
| EGF-containing fibulin-like extracellular matrix protein 1 (Extracellular | EFEMP1 FBLN3 |
| protein S1-5) (Fibrillin-like protein) (Fibulin-3) (FIBL-3) | FBNL |
| Multimerin-1 (EMILIN-4) (Elastin microfibril interface located protein | MMRN1 ECM |
| 4) (Elastin microfibril interfacer 4) (Endothelial cell multimerin) | EMILIN4 GPIA* |
| | MMRN |
| Trem-like transcript 1 protein (TLT-1) | TREML1 TLT1 |
| | UNQ1825/PRO3438 |
| Aprataxin and PNK-like factor (EC 3.1.—.—) (Apurinic-apyrimidinic | APLF C2ORF13 |
| endonuclease APLF) | PALF XIP1 |
| ATP-dependent RNA helicase TDRD9 (EC 3.6.4.13) (Tudor domain- | TDRD9 C14ORF75 |
| containing protein 9) | |
| Progesterone-induced-blocking factor 1 (PIBF) (Centrosomal protein of | PIBF1 C13ORF24 |
| 90 kDa) (CEP90) | PIBF |
| SPRY domain-containing SOCS box protein 2 (SSB-2) (Gene-rich | SPSB2 GRCC9 |
| cluster protein C9) | SSB2 |
| Keratin, type I cytoskeletal 23 (Cytokeratin-23) (CK-23) (Keratin-23) | KRT23 |
| Unconventional myosin-Va (Dilute myosin heavy chain, non-muscle) | MYO5A MYH12 |
| (Myosin heavy chain 12) | |

Having described specific embodiment of the present invention in detail above, it is to be understood that variants and modifications thereof falling within the spirit of the invention may become apparent to those skilled in this art, and the scope of this invention is to be determined by appended claims and their equivalents.

1. A method for isolating an apolipoprotein from a biological sample, comprising adding an organic solvent represented by R—CN (wherein R is a straight-chain or branched C₁-C₃ alkyl) to a biological sample comprising an apolipoprotein.

2. The method of claim 1, wherein the apolipoprotein is selected from the group consisting of ApoA-I, ApoA-II, ApoA-IV, ApoA-V, ApoB, ApoC-I, ApoC-II, ApoC-III, ApoC-IV, ApoD, ApoE, ApoF, ApoL1, ApoL2, ApoL3, ApoL4, ApoL5, ApoL6, Apo(a) and ApoM.

3. The method of claim 1, wherein the biological sample is selected from the group consisting of whole blood, plasma, and serum.

4. The method of claim 1, wherein the organic solvent is 40-80 v/v % acetonitrile.

5. A method for detecting an apolipoprotein in a biological sample, comprising isolating the apolipoprotein from the biological sample by performing the method of claim 1.

6. The method of claim 5, wherein the apolipoprotein is ApoC-III, and wherein the method is performed via multiple reaction monitoring (MRM).

7. The method of claim 5, wherein the method does not comprise digestion of the apolipoprotein.

8. A method for mass spectrometry of a target protein in a biological sample comprising adding a matrix composition comprising serum from a non-human animal as an active ingredient to the biological sample.

9. The method of claim 8, wherein the non-human animal is a ruminant.

10. The method of claim 9, wherein the mass spectrometry of the target protein is peptide-MRM (multiple reaction monitoring), and wherein the ruminant is a goat.

11. The method of claim 9, wherein the mass spectrometry of the target protein is protein-MRM, and wherein the ruminant is a bovine.

12. A method for mass spectrometry of a target protein in a biological sample comprising adding an internal standard (IS) composition comprising serum of a non-human animal as an active ingredient to the biological sample.

13. The method of claim 12, wherein the non-human animal is a ruminant.

14. The method of claim 13, wherein the ruminant is a goat or a bovine.

15. The method of claim 12, wherein the mass spectrometry is selected from the group consisting of matrix-assisted laser desorption/ionization time-of-flight (MALDI-TOF), sulfface enhanced laser desorption/ionization time-of-flight (SELDI-TOF), electrospray ionisation time-of-flight (ESI-TOF), multiple reaction monitoring (MRM), triple quadrupole mass spectrometry (QqQ MS), Composition characterized in that it is made using a mass spectrometry method selected from the group consisting of liquid chromatography-mass spectrometry (LC-MS) and liquid chromatography-mass spectrometry/mass spectrometry (LC-MS/MS).
